(19) United States
(12) Patent Application Publication
(10) Pub. No.: US 2026/0063619 A1
(43) Pub. Date: Mar. 5, 2026

(54) SAMPLE COLLECTION AND ANALYSIS SYSTEM

(71) Applicant: 3M INNOVATIVE PROPERTIES COMPANY, Saint Paul, MN (US)

(72) Inventors: Ramasubramani Kuduva Raman THANUMOORTHY, Woodbury, MN (US); Christopher R. KOKAISEL, Woodbury, MN (US); Joseph C. DINGELDEIN, Hudson, WI (US); Alan R. DOMBROWSKI, Woodbury, MN (US); Audrey A. SHERMAN, Woodbury, MN (US); Michael R. BERRIGAN, Oakdale, MN (US); Tonya D. BONILLA, Woodbury, MN (US)

(21) Appl. No.: 19/107,136

(22) PCT Filed: Aug. 29, 2023
(86) PCT No.: PCT/IB2023/058532

§ 371 Date: Feb. 27, 2025

Related U.S. Application Data
(60) Provisional application No. 63/402,832, filed on Aug. 31, 2022.

(51) Int. Cl. G01N 33/497; A61B 10/00; G01N 33/543; G01N 33/569

(52) U.S. Cl. CPC G01N 33/497; A61B 10/00; G01N 33/54388; G01N 33/54391; G01N 33/56983; A61B 2010/0006; A61B 2010/0087

(57) ABSTRACT

A sample collection system includes a housing comprising an air inlet constructed to receive an exhalation airflow; a porous sample collection media disposed within the housing; an airflow path extending from the air inlet through the porous sample collection media; a liquid reservoir in fluid communication with the porous sample collection media, constructed to direct liquid onto the porous sample collection media, the liquid reservoir including: a volume housing the liquid; an opening; and a removable tab sealing the opening; and an assay constructed to receive an eluted sample from the porous sample collection media. A method of collecting and testing a sample includes flowing exhalation air through the porous sample collection media to form a captured sample; releasing a metered dose of liquid onto the porous sample collection media to elute the sample onto the assay; and observing a test result on the assay.

### BACKGROUND

Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container.

Recently, an unprecedented need for rapid viral testing has arisen due to the COVID-19 pandemic. Attempts to control the pandemic require a massive expansion of testing for SARS-COV-2 virus in several different clinical and epidemiological contexts. Until recently, nasopharyngeal (NP) swabs were the United States Centers for Disease Control and Prevention's (CDC) preferred specimen type, as these specimens were thought to provide the most robust detection of patient infection. However, there are conflicting reports as to which of several specimen types bear the highest viral load.

Sensitivity is a complex issue, however, as detection in the upper airways (nasopharynx and oropharynx) is affected by multiple factors including duration of illness prior to testing, as well as the limit of detection (LoD) of the RT-PCR assay used. Availability of NP swabs and the resources to establish NP collection sites with specimen collection personnel have remained critical bottlenecks. To resolve these issues, healthcare systems have adopted multiple different strategies, including engaging industrial manufacturers to mass produce novel 3D-printed NP swabs, as well as evaluating different specimen types and alternative sample-collection strategies, such as saliva.

Assessment of nasal swabs and saliva is a rapidly growing area of interest, specifically because these specimen sample type involves a less invasive procedure than NP swabs. Accordingly, such samples can be self-collected by patients with a simple set of instructions, alleviating the need for highly trained medical personnel for specimen collection and reducing use of personal protective equipment (PPE) in short supply.

Many of the US Food and Drug Administration Emergency Use Authorization (FDA EUA) RT-PCR assays have approval for use of nasal swabs as a specimen type as well as saliva, but how well these samples perform compared to NP swabs remains unclear. To date, nasal-swab studies have shown conflicting results, with some researchers reporting similar test performance to NP swabs and others finding decreased sensitivity.

Currently available at-home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

The need exists for a simpler and cleaner sample collection and analysis system and easy elution of samples in a simple-to-use procedure. Further, a need exists for a sample collection and analysis system that can also capture and elute samples that have a low SARS-COV-2 viral load but are still capable of transmitting the virus to others. There is also a need for a more precise system to reduce human error and provide more repeatable a reliable test results.

### SUMMARY

There is a need for an inexpensive, simple to use, and reliable sample collection and analysis system that may be used by laypeople for testing for the presence of a target virus, target pathogen, or other target analyte, in a collected sample. The sample collection and analysis system may include a sample collection device for collecting a sample from exhalation airflow and a testing assay to determine the presence or absence of virus or other pathogen in the collected sample.

It is desirable to provide a system that includes both a sample collector device and rapid antigen testing in an integrated system. The integrated system may advantageously be self-contained and optionally sterile. A self-contained and sterile system may improve accuracy and reliability of pathogen testing due to the reduced contamination and background noise, unlike swabs and other test collection devices which may be contaminated upon use and/or during testing.

It is further desirable to provide a system which, after sample collection and optional testing, remains closed and self-contained to contain any potential virus or pathogen, and which may be safely disposed of among ordinary waste collection.

According to an embodiment, a sample collection and analysis system includes a housing comprising an air inlet constructed to receive an exhalation airflow; a porous sample collection media disposed within the housing; an airflow path extending from the air inlet through the porous sample collection media; a liquid reservoir in fluid communication with the porous sample collection media, constructed to direct liquid onto the porous sample collection media, the liquid reservoir comprising: a volume housing the liquid; an opening; and a removable tab sealing the opening; and an assay constructed to receive an eluted sample from the porous sample collection media. The liquid reservoir may be immediately adjacent to the air inlet. The liquid reservoir may be distanced from the air inlet.

The removable tab may be positioned between the volume and the porous sample collection media. The removable tab may be a pull tab. The removable tab may comprise a first portion disposed between the volume and the porous sample collection media and a second portion extending outside of the housing and forming a pull tab.

The porous sample collection media defines a surface area and the volume of the liquid reservoir divided by the surface area may be in a range from 10 μL/cm2 to 400 μL/cm2, or from 10 μL/cm2 to 250 μL/cm2. The volume may be in a range of 50 μL to 1000 μL. The liquid reservoir may house from 50 μL to 1000 μL of the liquid.

The housing may include a first end and an opposing second end, wherein the air inlet is disposed adjacent the first end, wherein an air outlet is disposed adjacent the second end, and wherein the airflow path extends from the air inlet to the air outlet. The airflow path may extend along a length of the assay. The porous sample collection media may comprise a nonwoven filtration layer having an electrostatic charge. The nonwoven filtration layer is hydrophobic. The liquid may be an aqueous solution comprising a surfactant.

The assay may be constructed to detect presence of a virus or other pathogen in a collected sample. The assay may be a lateral flow assay, a vertical flow assay, or a colorimetric indicator.

A method of collecting and testing a sample includes: flowing exhalation air through a porous sample collection media to form a captured sample, the porous sample collection media being disposed within a sample collection device housing; releasing a metered dose of liquid onto the porous sample collection media from a liquid reservoir disposed within the sample collection device housing by pulling on a pull tab, the metered dose of liquid eluting the captured sample onto an assay disposed within the sample collection device housing; and observing a test result on the assay. The flowing of exhalation air may comprise blowing into a mouthpiece or nosepiece on the sample collection device. The observing of the test result comprises observing a positive result if a target virus or other target pathogen is present or negative result if a target virus or other target pathogen is absent.

The porous sample collection media is constructed to capture a sample of viruses, pathogens, or other analytes from exhalation airflow. The porous sample collection media may be made of nonwoven material. The nonwoven material may include polylactic acid, polypropylene, or a combination thereof. The nonwoven material may carry an electrostatic charge. The assay may be a lateral flow assay or a vertical flow assay. The assay is constructed to determine the presence or absence of a target virus, pathogen, or analyte in the collected sample. The assay may be constructed to display a positive or negative result regarding the presence of a target virus, pathogen, or analyte.

The assay may define a strip of material with a length, and wherein the length is parallel to a longitudinal axis of the second part. The assay may define a strip of material with a length, and wherein the length is transverse to a longitudinal axis of the second part.

According to an embodiment, a kit includes the sample collection and analysis system and instructions for collecting a sample and testing the sample using the assay. The instructions may include instructions to: exhale along the airflow path to capture a sample in the porous sample collection media; move or remove the removable tab to release a metered dose of liquid onto the porous sample collection media; and observe a test result in a result display of the assay. The instructions may further include instructions to read a test result display of the assay using an electronic reader.

### BRIEF DESCRIPTION OF FIGURES

FIG. 1A is a top view of a sample collection and analysis system according to an embodiment.

FIG. 1B is a cross-sectional side view of the system of FIG. 1A.

FIG. 2A is a partial cross-sectional perspective view of the system of FIG. 1A.

FIG. 2B is a partial cross-sectional perspective view of the system of FIG. 2A with the pull tab being partially removed.

FIG. 2C is a perspective view of a liquid capsule incorporated in the sample collection and analysis system of FIG. 1A.

FIG. 2D is a side view of the liquid capsule of FIG. 2C.

FIG. 3A is a partial cross-sectional top perspective view of the system of FIG. 1A with an alternative pull tab arrangement.

FIG. 3B is a partial cross-sectional bottom perspective view of the system of FIG. 3A.

FIG. 4A is a perspective view of a sample collection and analysis system according to an alternative embodiment.

FIG. 4B is a cross-sectional side view of the system of FIG. 4A.

FIG. 5A is a partial cross-sectional perspective view of the system of FIG. 4A.

FIG. 5B is a partial cross-sectional perspective view of the system of FIG. 5A with the pull tab being partially removed.

FIG. 6A is a top view of a sample collection and analysis system according to an embodiment.

FIG. 6B is a side view of the system of FIG. 6A.

FIG. 6C is a cross-sectional perspective view of the system of FIG. 6A.

FIG. 6D is a partial cross-sectional side view of the system of FIG. 6A.

FIG. 7A is a perspective view of the bottom part and liquid reservoir of the system of FIG. 6A.

FIG. 7B is a top view of the bottom part of the system of FIG. 6A.

FIG. 8A is a perspective view of a sample collection and analysis system according to an embodiment.

FIG. 8B is a cross-sectional side view of the system of FIG. 8A.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

All headings provided herein are for the convenience of the reader and should not be used to limit the meaning of any text that follows the heading, unless so specified.

The term “i.e.” is used here as an abbreviation for the Latin phrase id est, and means “that is,” while “e.g.” is used as an abbreviation for the Latin phrase exempli gratia and means “for example.”

The term “about” is used here in conjunction with numeric values to include normal variations in measurements as expected by persons skilled in the art and is understood have the same meaning as “approximately” and to cover a typical margin of error, such as ±5% of the stated value. Moreover, unless otherwise indicated, all numbers expressing quantities, and all terms expressing direction/orientation (e.g., vertical, horizontal, parallel, perpendicular, etc.) in the specification and claims are to be understood as being modified in all instances by the term “about.” Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The terms “downstream” and “upstream” refer to a relative position based on a direction of exhalation airflow through the device. For example, the upstream-most element of the device is the air inlet element, and the downstream-most element of the device is the exhalation outlet element.

### DETAILED DESCRIPTION

The present disclosure relates to a sample collection analysis system. The present disclosure relates to a bioaerosol collection device. The present disclosure further relates to a system that includes both sample collection and testing capabilities.

The sample collection and analysis system of the present disclosure includes a sample collection device. The sample collection device includes a porous sample collection media disposed within the device housing and along an airflow path defined by the device housing. The porous sample collection media is constructed to capture viruses, pathogens, or other analytes, carried in an exhalation airflow. The system may further include a sample testing assay. A metered dose of liquid is passed through the porous sample collection media and carries away pathogen or virus that may be bound to the porous sample collection media, forming an eluent. The metered dose of liquid may be provided in a reservoir provided within the housing. The eluent may further flow onto the assay and be analyzed using the assay.

In particular, the present disclosure relates to a sample collection device including a housing extending from an air inlet to an air outlet. The housing defines an airflow path from the air inlet to the air outlet. The air inlet is configured to receive an exhalation airflow. A porous sample collection media is disposed within the housing and along the airflow path.

The housing may include a mouthpiece or nosepiece or another structure that facilitates breathing into the air inlet. For convenience, reference is made here to a mouthpiece but it should be understood that the structure may also be a nosepiece or other suitable structure. The mouthpiece may be aligned with the inlet opening. The mouthpiece may help a user direct exhalation airflow onto the porous sample collection media. The mouthpiece may be integral with the housing or may be removably coupled with the housing.

The reservoir containing the metered dose of liquid (referred to here as the liquid reservoir) may be disposed within the housing. The liquid reservoir may be formed by the housing itself or may be provided as a liquid capsule disposed within the housing. The liquid reservoir may be constructed to release a metered dose of liquid onto the porous sample collection media. The reservoir (e.g., liquid capsule) may be prepared from any suitable material, such as polyethylene (PE), polypropylene (PP), polyester terephthalate (PET), or the like. A removable tab may removably seal the liquid reservoir. The removable tab may be sealed onto the opening of the liquid reservoir by a peelable seal. The peelable seal may be formed, for example, by a heat seal, a suitable adhesive, a gasket, or a combination thereof. The removable tab may be constructed of a material that is compatible with the reservoir (e.g., liquid capsule) material. In some embodiments, the removable tab may have a layered construction, where the layer contacting the reservoir (e.g., liquid capsule) is made of a material that is compatible with the reservoir (e.g., liquid capsule) material. The removable tab may also include a barrier layer. For example, the removable tab may include a metalized layer, such as an aluminum layer, to provide a fluid and vapor barrier. Exemplary suitable materials for the removable tab, available from the 3M company, include 3M 9792R Single-Coated Aluminum; 3M 9793R SC Polyolefin; 3M 9794R SC Clear PET; 3M 9795R SC Advanced Polyolefin; 3M 9964 SC Clear PET; and 3M 9965 Double-Coated White PET [2 Liners]. Suitable adhesives include adhesives that seal the reservoir such that the liquid does not flow out prematurely but allow the tab to be removed by a user. In some embodiments, the removable tab has a weaker (or “soft”) seal on one or more sides (e.g., on three sides) and a stronger (or “hard”) seal on at least one side. This may allow a user to easily peel the tab off of the liquid reservoir opening to release the liquid, while leaving the tab attached to the device on one side. For example, the peelable seal may have a peel force of 1.81 kgf (4 lbf) or less, or from 0.45 kgf (1 lbf) to 0.9 kgf (2 lbf). The strength of the seal may depend on the material and width and design of the seal.

The liquid dispensed onto the porous sample collection media may be an aqueous liquid. The liquid may be a buffer solution. The liquid may be an aqueous buffer solution. The liquid may be a saline solution. The liquid may include a surfactant. A “surfactant” is generally understood to mean a molecule that can be added to a solution to reduce the surface tension of the solution. The liquid may be formulated to have a surface tension that facilitates release from the reservoir, as well as flow through or across the porous sample collection media and onto the assay. For example, the surface tension of the liquid may be lower than that of water. The liquid may have a contact angle of greater than 90 degrees when measured on the porous sample collection media. The liquid may be a saline solution including a surfactant. The liquid (e.g., a buffer or a saline solution) may include from 0.1 wt-% or more or 0.5 wt-% or more, and up to 1 wt-% or up to 2 wt-% of surfactant. The metered dose of liquid may have a volume of 50 μL or greater, 100 μL or greater, 150 μL or greater, 200 μL or greater, or 250 μL or greater. The metered dose of liquid may have a volume of 1200 μL or less, 1000 μL or less, 750 μL or less, 500 μL or less, or 400 L or less. The metered dose of liquid may have a volume of 50 μL to 1000 μL or 100 μL to 500 μL.

In some embodiments, the system includes two or more liquid reservoirs, each liquid reservoir being constructed to deliver liquid onto the porous sample collection media. The liquid contained in the two or more liquid reservoirs may be different from one another. For example, one liquid reservoir may contain a buffer solution to elute the sample and another liquid reservoir may contain a reagent used to analyze the sample. The liquids from the two or more liquid reservoirs may be released simultaneously or in succession.

The liquid may be applied onto the loaded porous sample collection media. The liquid may travel through the surface and thickness of the loaded porous sample collection media and flow off of the porous sample collection media carrying with it any virus, pathogen, or other analyte, that was present on the loaded porous sample collection media. This loaded liquid may then be received by the assay and tested for the presence of the virus, pathogen, or other analyte of interest.

According to an embodiment, a sample collection and analysis system includes the sample collection device described above and an assay configured to receive the metered dose of liquid (e.g., the eluent) from the porous sample collection media. The assay may further be configured to analyze the analyte of interest, such as a virus, other pathogen, or other analyte. The assay may be integral with the sample collection device. The assay may form a unitary element with the housing of the sample collection device.

The user may exhale into the sample collection device and load the porous sample collection media with a sample of the exhalation airflow to form a loaded porous sample collection media. For example, the user may exhale through the air inlet or through the mouthpiece or nosepiece. The housing may be constructed such that by exhaling through the single opening, air inlet, mouthpiece, or nosepiece, the exhalation airflow passes through the porous sample collection media. The porous sample collection media is constructed to capture viruses, other pathogens, or other analytes, from the exhalation airflow. The user may then release a metered dose of liquid from the liquid reservoir to apply the liquid to the loaded porous sample collection media and to elute the captured sample onto the assay. The user may test the eluent for the presence of a virus, pathogen, or other analyte using the assay. The testing may take place with the loaded porous sample collection media in place in the sample collection and analysis system.

According to an embodiment, the sample collection and analysis system forms a singular self-contained unit. Providing a self-contained unit allows for convenient shipping and transportation of the sample collection and analysis system and for disposal after use. The self-contained unit may have a compact size and may be conveniently carried in a pocket or purse. The self-contained unit may be safely disposed of after use among ordinary waste disposal.

The assay may be a separate element from the sample collection device. The assay may be configured to attach to the sample collection device. The sample collection device may include a receptacle for receiving at least a portion of the assay. The sample collection device may include a receptacle for receiving the entire assay. The assay may be a replacement element with the sample collection device.

Similarly, the liquid capsule may be a separate element from the sample collection device and may be configured to be received by the housing. The housing may include a receptacle (e.g., reservoir) for receiving the liquid capsule. The liquid capsule may be a replacement element with the sample collection device. One illustrative metered fluid dose element is commercially available from 3M Company (St. Paul MN, U.S.A.) under the trade designation CUROS.

The assay included in the sample collection and analysis system may be any suitable assay. The assay may be constructed to determine the presence or absence of a target virus, pathogen, or analyte in the collected sample. In some embodiments, the assay is a lateral flow assay (“LFA”), a vertical flow assay (“VFA”), or a colorimetric indicator. LFAs and VFAs are generally paper-based platforms for the detection and quantification of analytes in complex mixtures, including biological samples such as saliva, urine, etc. LFAs and VFAs are typically easy to use and can be used both by professionals in a health care setting or laboratory as well as by lay persons at home. Typically, a liquid sample is placed on the assay in a sample receiving region and is wicked by capillary flow along the device to a test region. LFAs and VFAs are typically based on antigens or antibodies that are immobilized in the test region and that selectively react with the analyte of interest. The result is typically displayed within 5 to 30 minutes. LFAs and VFAs can be tailored for the testing of a variety of viruses and other pathogens, as well as many other types of analytes. According to an embodiment, the assay used in the sample collection and analysis system of the present disclosure is constructed for the detection of a target virus, target pathogen, or other target analyte. According to an embodiment, the assay used in the sample collection and analysis system of the present disclosure is constructed for the detection of a target virus, target pathogen, or other target analyte, that may be present in the exhalation air flow of a subject.

Examples of commercially available LFAs that may be included in the sample collection and analysis system include AccessBio CARESTART™ COVID-19 Antigen Home Test, Abbott BINAXNOW™ COVID-19 Antigen Self Test, and Quidel QUICKVUE® At-Home OTC COVID-19 Test.

Examples of colorimetric indicators include LFA colorimetric readers utilizing image sensors, such as a charge-coupled device (CCD) or complementary metal oxide semiconductor (CMOS). Such devices are useful, at least in part, due to their simple structure and small size. When the device is used, the LFA develops a test line, which is an aggregate of labeled particles, antigens, and antibodies. An image sensor-based LFA reader acquires an image of the test line analyzes the pixel intensity of the test line, which changes according to the concentration of the target analyte.

According to an embodiment, the porous sample collection media is suitable for exhalation through the media. That is, the porous sample collection media has sufficient porosity to allow exhalation through the media. As used here, the term “porosity” refers to a ratio of open space in the media to the amount of volume taken by the media material itself. A media with high porosity has more open space and, therefore, allows higher flow with a lower pressure drop.

According to an embodiment, the porous sample collection media is a nonwoven material carrying an electrostatic charge. The electrostatic charge may enable capturing pathogens, viruses, or other analytes from an exhalation airflow. In some cases, the porous sample collection media may be a hydrophobic nonwoven material. In other cases, the porous sample collection media may be a hydrophilic nonwoven material. The porous sample collection media may be a hydrophobic nonwoven material carrying an electrostatic charge configured to capture pathogens, viruses, or other analytes from an exhalation airflow. The porous sample collection media may be a hydrophilic nonwoven material carrying an electrostatic charge configured to capture pathogens, viruses, or other analytes from an exhalation airflow. The term “hydrophobic” refers to a material having a water contact angle of 90 degrees or greater, or from about 90 degrees to about 170 degrees, or from about 100 degrees to about 150 degrees. The term “hydrophilic” refers to a material having a water contact angle of less than 90 degrees. Water contact angle is measured using ASTM D5727-1997 Standard test method for surface wettability and absorbency of sheeted material using an automated contact angle tester.

The porous sample collection media may be formed of any suitable material that is capable of capturing viruses, pathogens, or other analytes from exhalation airflow and releasing the captured viruses, pathogens, or other analytes upon being contacted with an eluent, such as a saline solution. The porous sample collection media may be formed of polymeric material. The porous sample collection media may be formed of a polyolefin. Examples of suitable polyolefins include polypropylene, polylactic acid, and the like, and a combination thereof. In one embodiment the porous sample collection media is formed of polypropylene. In one embodiment the porous sample collection media is formed of polylactic acid. One illustrative porous sample collection media is commercially available from 3M Company (St. Paul MN, U.S. A.) under the trade designation FILTRETE Smart MPR 1900 Premium Allergen, Bacteria & Virus Air Filter Merv 13.

The porous sample collection media may have a thickness (orthogonal to the major plane) of 200 μm or greater or 250 μm or greater. The porous sample collection media may have a thickness of 750 μm or less or 1000 μm or less. The porous sample collection media may have a thickness of in a range from 200 μm to 1000 μm, or from 250 μm to 750 μm. The porous sample collection media may have major plane surface area (of one side) of 1 cm² or greater or 2 cm² or greater. The porous sample collection media may have major plane surface area of 3 cm² or less or 4 cm² or less. The porous sample collection media may have major plane surface area in a range from 1 cm² to 4 cm², or 2 cm² to 3 cm².

The housing may be formed of a rigid material, such as plastic or a paper-based material such as cardboard or cardstock. In some embodiments, the housing is made of plastic. The housing may be made of a material that does not absorb any of the liquid or eluent. For example, the housing may be made of a hydrophobic material. In some embodiments, at least a portion of the housing is transparent. For example, the housing may include transparent material in an area of a result display of the assay. The housing may include a viewing window (either transparent material or an opening) in the area of the result display. In some cases, the entire housing may be made of a transparent material. The housing may further include a cover or sealing layer constructed to prevent contamination before or after use of the system. The cover or sealing layer may be removable (e.g., may be removed before use). The cover or sealing layer may be closable and/or re-closable (e.g., may be closed after use).

The housing may include a pre-filter or screen disposed in the airflow path in front (upstream) of the porous sample collection media. The screen may be constructed to catch larger particles (larger than viruses or pathogens) and prevent such particles from reaching the porous sample collection media. The exhalation airflow passes through a thickness of the pre-filter or screen. The pre-filter or screen at least partially occludes the air flow path. In some cases, the pre-filter or screen may have a major plane that is orthogonal to the direction of the exhalation airflow passing through the thickness of the pre-filter or screen. The pre-filter or screen may be a non-woven layer configured to filter out larger particles from the exhalation airflow passing through the pre-filter or screen. In some cases, the pre-filter or screen may be a non-woven layer that does not have an electrostatic charge. In some embodiments, the pre-filter or screen does not capture significant amounts of viral material, pathogen material, or other analyte material, and instead allows them to transmit through the pre-filter or screen. In some embodiments, the pre-filter or screen is made of or includes at least one of a plastic mesh, a woven net, a needle-tacked fibrous web, a knitted mesh, an extruded net, and/or a carded or spunbond coverstock.

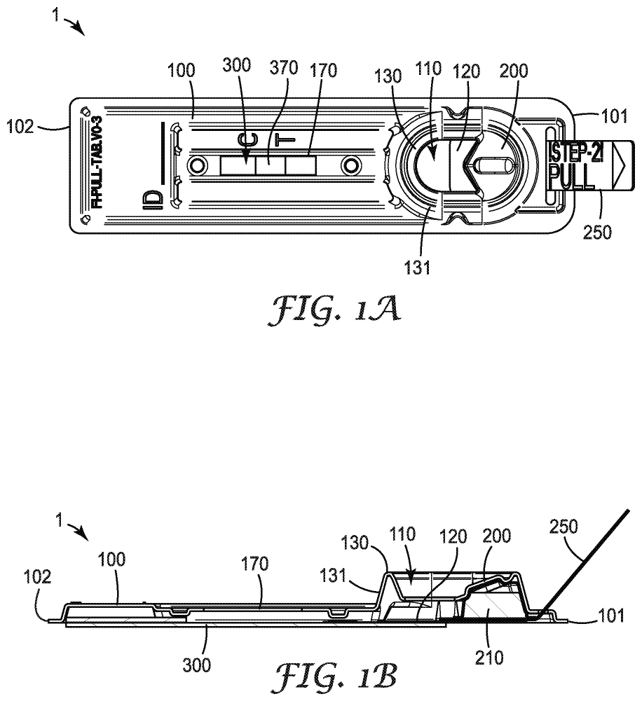

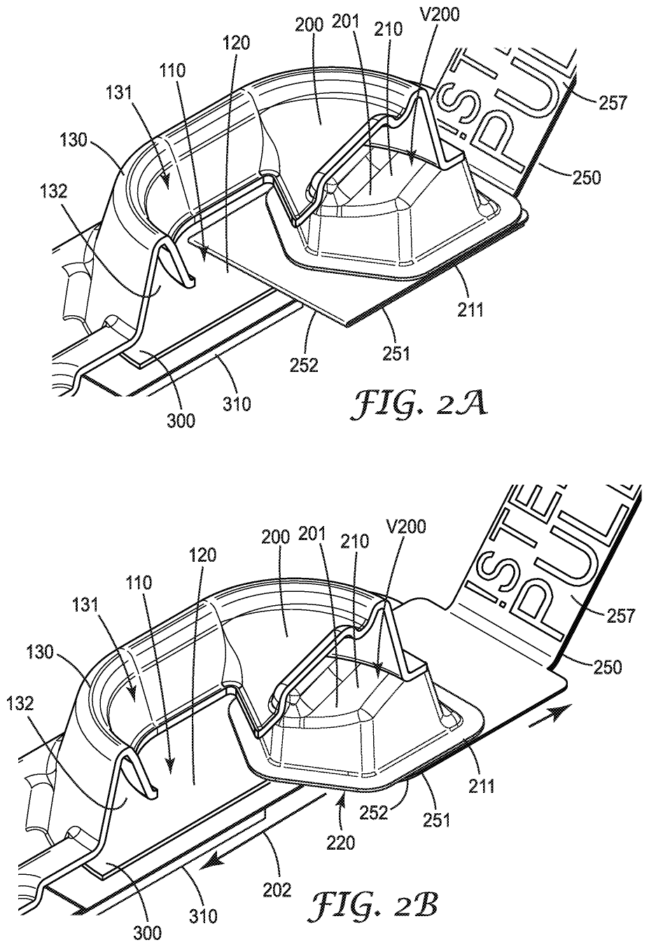

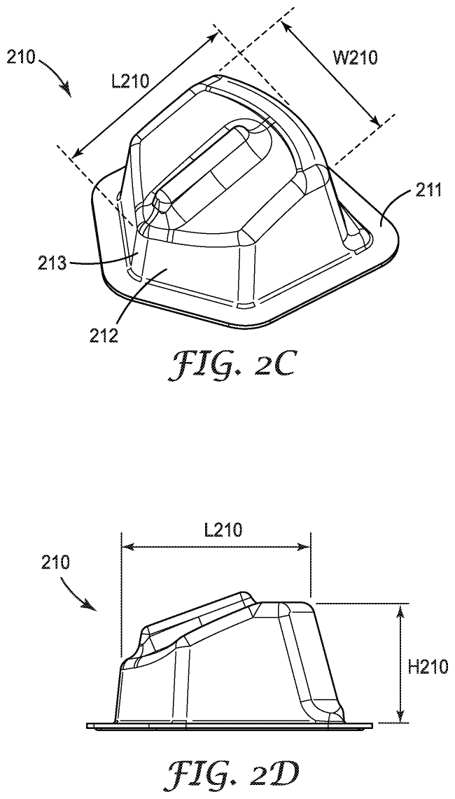

Referring now to FIGS. 1A-2B, an illustrative example of a sample collection and analysis system 1 is shown. According to an embodiment, the system 1 includes both sample collection and testing capabilities. The system is shown in FIGS. 1B-2B with its bottom cover removed to better illustrate the internal components. As seen in FIGS. 1A and 1B, the system 1 has a housing 100 extending from a first end 101 to a second end 102. The housing 100 has an air inlet 131 constructed to receive an exhalation airflow. The air inlet 131 may be provided as part of a mouthpiece 130, as shown. Alternatively, the housing 100 may include a nosepiece or another structure associated with the air inlet 131. A porous sample collection media 120 is disposed within the housing. The housing 100 defines an airflow path 110 extending from the air inlet 131 and through the porous sample collection media 120. The porous sample collection media 120 is constructed and arranged to capture viruses, other pathogens, or other analytes from exhalation airflow flowing through the airflow path 110.

According to an embodiment, the porous sample collection media 120 at least partially occludes the airflow path 110. The porous sample collection media 120 may completely occlude the airflow path. Exhalation airflow may pass through the thickness of the porous sample collection media 120. The porous sample collection media 120 may have a major plane that is orthogonal to the direction of the exhalation airflow passing through the thickness of the porous sample collection media 120.

The porous sample collection media 120 may be a nonwoven material configured to filter virus, pathogens, or other analytes from an exhalation airflow. The porous sample collection media 120 may be a nonwoven material having an electrostatic charge configured to filter virus, pathogens, or other analytes from an exhalation airflow. The porous sample collection media 120 may be a hydrophobic nonwoven material configured to filter virus, pathogens, or other analytes from an exhalation airflow. The porous sample collection media 120 may be a hydrophobic nonwoven material having an electrostatic charge configured to filter virus, pathogens, or other analytes from an exhalation airflow. The porous sample collection media 120 may be formed of polymeric material. Suitable materials for the porous sample collection media 120 are discussed above.

The porous sample collection media 120 may have a thickness (orthogonal to the major plane) in a range from 200 μm to 1000 μm, or from 250 μm to 750 μm. The porous sample collection media 120 may have major plane surface area in a range from about 1 cm² to about 4 cm², or about 2 cm² to about 3 cm².

The housing 100 further includes a liquid reservoir 200. According to an embodiment, the liquid reservoir 200 has a volume V200 housing a metered dose of liquid 201 for eluting a sample captured by the porous sample collection media 120. The liquid reservoir 200 may be in fluid communication with (e.g., adjacent or immediately adjacent) the porous sample collection media 120 such that when the liquid 201 is released from the liquid reservoir 200, it may flow onto the porous sample collection media 120.

The liquid reservoir 200 defines an opening 220, which may be removably sealed by a removable tab 250. A removable tab 250 is positioned between the opening 220 and the porous sample collection media 120.

In the embodiment shown, the liquid reservoir 200 overlaps the porous sample collection media 120. Alternatively, the liquid reservoir 200 may be in fluid communication with (e.g., adjacent or immediately adjacent) the porous sample collection media 120 such that liquid 201 from the liquid reservoir 200 flows onto the porous sample collection media 120. The system 1 may be provided with an additional wicking pad arranged to wick the liquid 201 from the opened liquid reservoir 200 to the porous sample collection media 120.

In the embodiment shown in FIGS. 1A-2B, the liquid reservoir 200 is formed as part of the mouthpiece 130. The mouthpiece 130 may be formed by a hollow ring 132 around the air inlet 131. The liquid reservoir 200 may be formed in an expansion of the hollow ring 132.

The liquid reservoir 200 may include a capsule 210 disposed within the liquid reservoir 200, housing the liquid 201. The volume V200 of the liquid reservoir 200 may be defined by the capsule 210. The liquid 201 may be dispensed into the capsule 210 and the capsule 210 may be removably sealed with the tab 250. That is, instead of being sealed directly onto the housing 100, the tab 250 may be sealed onto the capsule 210. The sealed capsule 210 may be placed inside the liquid reservoir 200 and the system 1 may be assembled. The capsule 210 may be shaped to fit snugly inside and follow the contours of the liquid reservoir 200 formed on the housing 100. The capsule 210 may include a lip 211. The liquid reservoir 200 or the housing 100 may contain corresponding mating features, such as protrusions or detents, to facilitate coupling the liquid capsule 210 with the liquid reservoir 200. The lip 211 may facilitate sealing the tab 250 onto the capsule 210.

The removable tab 250 includes a first portion 251 and a second portion 257. The first portion 251 may be a sealing portion disposed against (e.g., sealed onto) the lip 211 (or the opening 220 if the system 1 does not include a capsule 210). The first portion 251 may be folded over itself and may include a fold 252, as shown in FIG. 2A. The second portion 257 may extend out from the housing 100 and may form a pull tab. A user may pull on the second portion 257 to at least partially slide the tab 250 from between the liquid reservoir 200 (e.g., capsule 210) and the porous sample collection media 120, as shown in FIG. 2B. The peel angle of the pull tab may be, for example, between 30-180 degrees. Pulling on the second portion 257 may unseal and at least partially expose the opening 220 to allow the liquid 201 to flow (arrow 202) onto the porous sample collection media 120. Folding the removable tab 250 enables the pull tab to be simply pulled rather than being sheared. The removable tab 250 may be sealed to the liquid reservoir 200, for example, by a heat seal, a suitable adhesive, a gasket, or a combination thereof.

Alternatively, the liquid 201 may be disposed directly in the liquid reservoir 200. In such embodiments, the opening of the liquid reservoir 200 is sealed by the tab 250.

The liquid reservoir 200 and/or the capsule 210 may be formed to facilitate liquid flow from the liquid reservoir 200 and/or capsule 210. For example, the capsule 210 may have a height to width ratio that facilitates (e.g., does not interfere with) liquid flow. As shown in FIGS. 2C and 2D, the capsule 210 has a height H210 and a width W210. The height H210 may be the vertical dimension when the system 1 is held horizontally, e.g., the length of the housing 100 is held in a horizontal orientation with the airflow inlet 131 facing upward. The height H210 may be the greatest height of the interior of the capsule 210. The width W210 may be the horizontal dimension, orthogonal to the vertical dimension. The width W210 may be the greatest width of the interior of the capsule 210 from a side wall to an opposing side wall. The liquid reservoir and/or capsule 210 may also have a length L210, orthogonal to both the height H210 and the width W210. At least one of, or preferably both, the width W210 and the length L210 is greater than the height H210. The height H210 (horizontal dimension) to width W210 (vertical dimension) ratio may be 1 or less, 0.9 or less, 0.8 or less, or 0.75 or less. The height H210 to width W210 ratio may be 0.3 or greater, 0.4 or greater, or 0.5 or greater. The height H210 to width W210 ratio may be from 0.5 to 0.75.

The side wall 212 facing the second end 102 of the housing may be angled or faceted and include a point 213 extending toward the second end 102 of the housing. The point 213 helps to lower the peel force needed to peel off the removable tab 250. The point 213 also provides a controlled release of the metered dose of liquid as the opening size gradually increases starting from the point 213.

The system 1 further includes an assay 300 disposed within the housing 100 and constructed to receive an eluted sample from the porous sample collection media 120. According to an embodiment, the assay 300 is in direct contact with the porous sample collection media 120. For example, a part of the assay 300 may overlap with the porous sample collection media 120, as shown in FIGS. 1B-2B. The liquid 201 with the eluted sample may be wicked from the porous sample collection media 120 onto a sample receiving area 310 of the assay 300. The eluted sample may be further wicked onto a test area 320. The assay 300 may be a flow assay, such as a lateral flow assay or a vertical flow assay. The assay 300 may be configured to detect a virus or other pathogen or analyte. The assay 300 may include a test result display 370 to indicate the presence or absence of a virus or other pathogen or analyte. The housing 100 may include a corresponding result viewing window 170 through which the test result display 370 may be viewed.

In some embodiments, the volume of the metered dose of liquid 201 is relative to the surface area of the porous sample collection media 120. The porous sample collection media 120 defines a major surface area and the metered dose of liquid 201 defines a volume, and the volume divided by the surface area may be in a range from 10 μL/cm² to 400 μL /m², or from 10 μL/cm² to 250 μL/cm², or from 50 μL/cm² to 150 μL/cm². In some embodiments, the metered dose of liquid 201 defines a volume in a range from 50 μL to 1000 LL or 100 μL to 500 μL.

The liquid 201 dispensed from the liquid reservoir 200 may be an aqueous liquid. The liquid 201 may be an aqueous buffer solution. The liquid 201 may be an aqueous liquid with a surfactant. The liquid 201 may be saline solution. The liquid 201 may be a saline solution comprising a surfactant. The liquid 201 may be a saline solution comprising from 0.5% to 2% surfactant by weight.

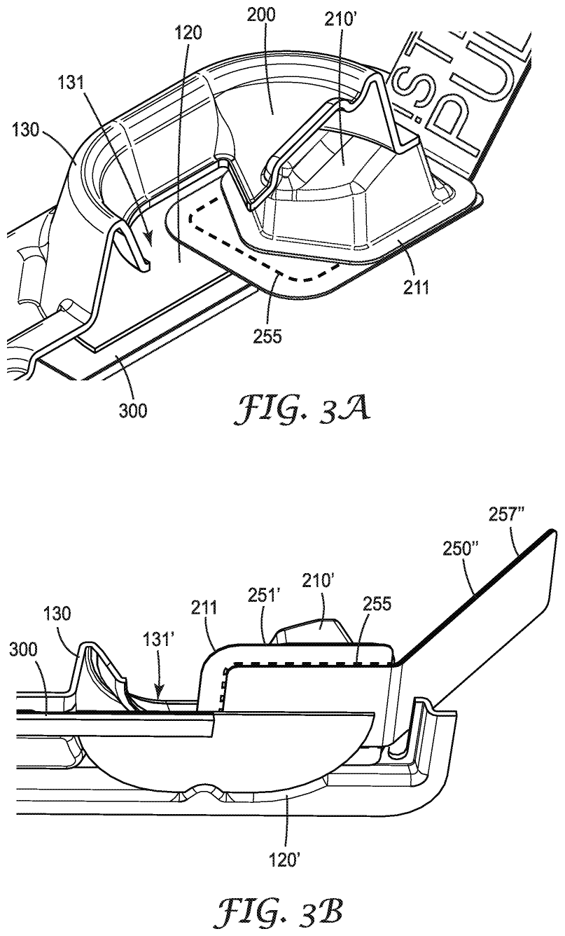

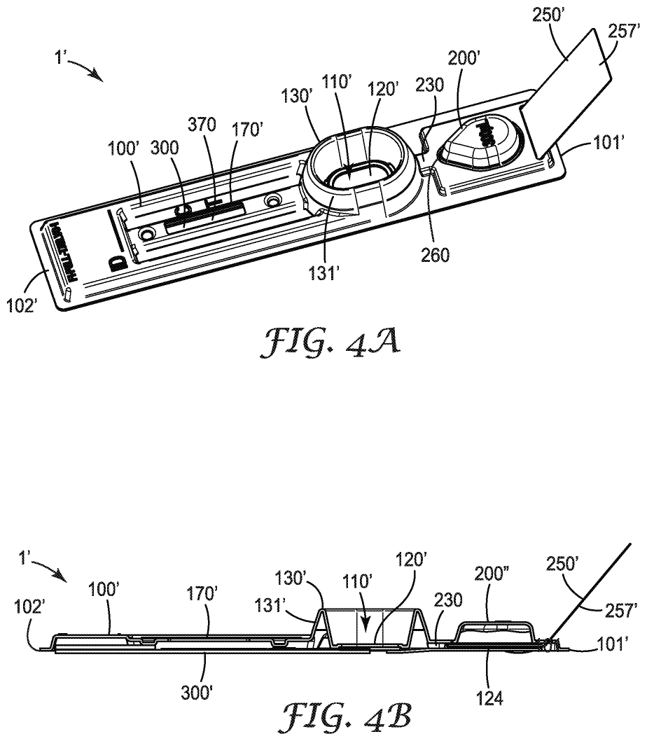

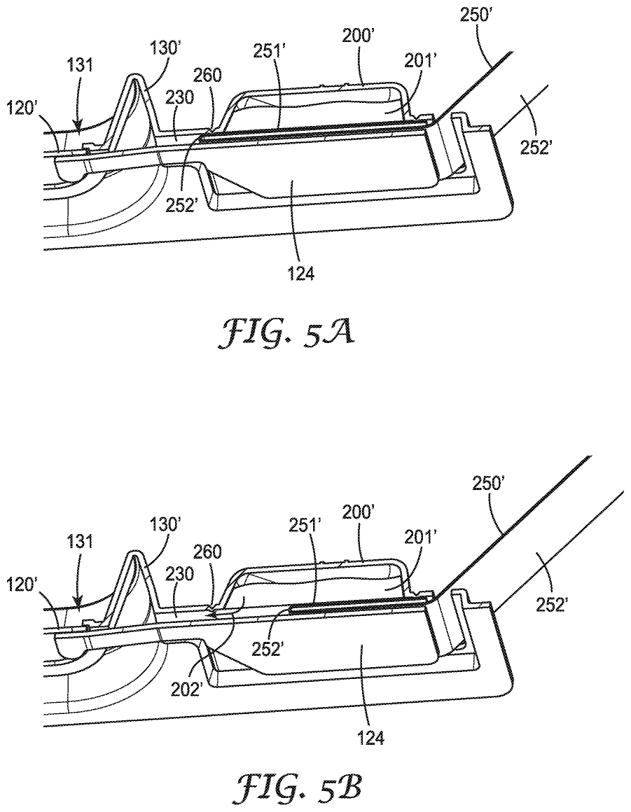

In some embodiments, instead of being folded over itself, the tab 250″ may include perforations 255 that facilitate removing the tab 250″, as shown in FIGS. 3A-3B. The system is shown in FIG. 3B with its bottom cover removed to better illustrate the internal components. The perforated tab 250″ includes a first portion 251″ and a second portion 257″. The first portion 251″ may form a sealing portion and is sealed onto the capsule 210′. Although the perforated tab 250″ is shown as being used with the system 1 of FIGS. 1A-1B, it may be combined with other configurations of the liquid reservoir, as well, such as the one shown in FIGS. 4A-5B. The first portion 251″ may be directly sealed onto liquid reservoir 200′. The perforations 255 may be placed along the perimeter of the first portion 251″, around the opening 220. When a user pulls on the second portion 257″ (pull tab), the tab 250″ separates along the perforations 255 to release the liquid 201 in the liquid reservoir 200 (e.g., capsule 210′).

In another embodiment, shown in FIGS. 4A-5B, the liquid reservoir 200′is distanced away from the air inlet 131 (e.g., mouthpiece 130). The system l′ is shown in FIGS. 5A and 5B with its bottom cover removed to better illustrate the internal components. The liquid reservoir 200′may be connected to the airflow path 110′by a channel 230. The channel 230 may facilitate wicking or flowing of the liquid 201 from the liquid reservoir 200′onto the porous sample collection media 120′.

In the embodiment shown, the liquid 201 is disposed directly in the liquid reservoir 200′formed by the housing 100′(without incorporating a capsule). The liquid reservoir 200′is sealed by the tab 250′. The liquid reservoir 200′is surrounded by a sealing groove 260. A first portion 251′of the tab may form a sealing portion and be sealed against the sealing groove 260. The tab 250′may be folded against itself along fold 252′. A second portion 257′of the tab extends out of the housing 100′. The second portion 257′may form a pull tab.

In the embodiment shown, a wicking pad 124 is arranged to wick the liquid 201 from the opened liquid reservoir 200′to the porous sample collection media 120′. The wicking pad 124 may be positioned directly adjacent (e.g., below) the liquid reservoir 200′. The wicking pad 124 may extend from below the liquid reservoir 200′to the porous sample collection media 120′.

The rest of the system 1′may be similar to the system 1 described with respect to FIGS. 1A-2B.

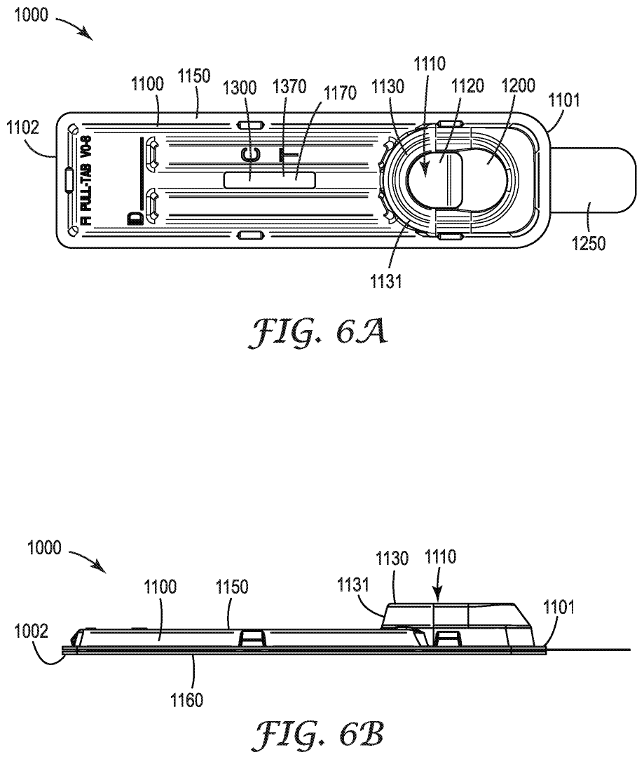

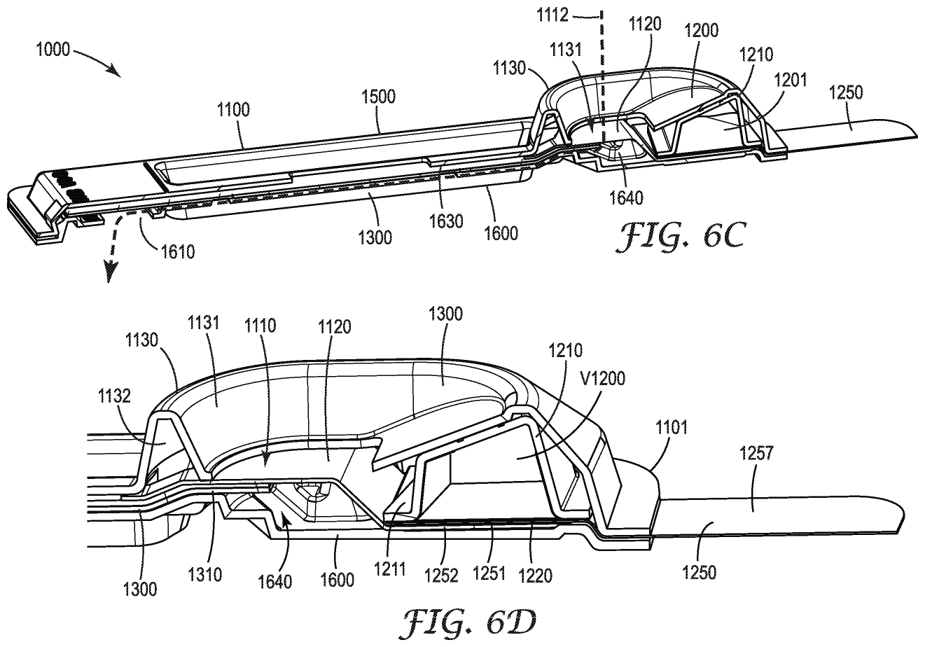

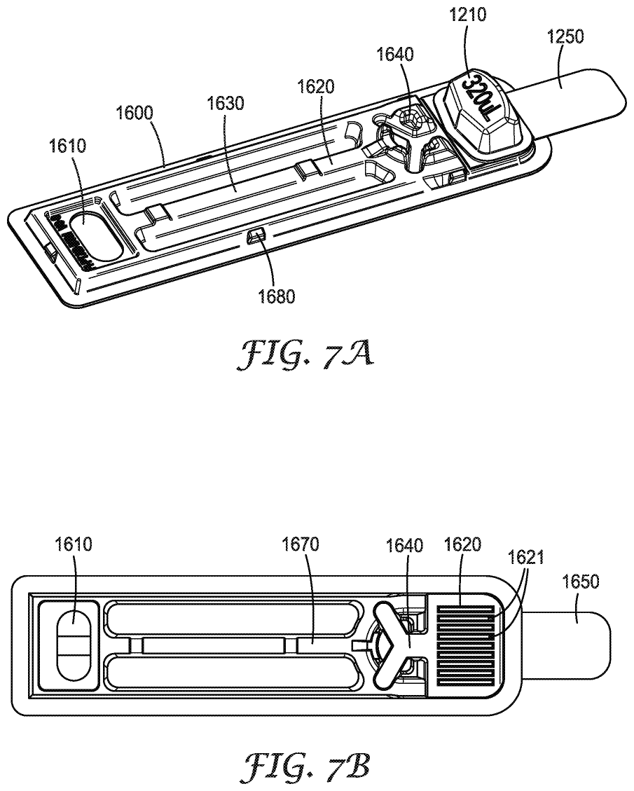

In some embodiments, an airflow channel 1110 extends longitudinally through the sample collection and analysis system 1000, as shown in FIGS. 6A-7B. The system 1000 includes a housing 1100 with a first end 1101 and opposing second end 1102, and an airflow inlet 1131 in a mouthpiece 1130. A porous sample collection media 1120 and an assay 1300 are disposed within the housing 1100.

The housing includes a liquid reservoir 1200 that may be similar to the liquid reservoir 200 of the system 1 of FIG. 1A, or alternatively similar to the liquid reservoir 200′of the system′ of FIG. 4A. A removable tab 1250 may be used to removably seal the liquid reservoir 1200. The assay 1300 may include a test result display 1370, which may be viewed through a result viewing window 1170 in the housing 1100.

The airflow channel 1110 may extend from the airflow inlet 1131 to an airflow outlet 1610. The airflow inlet 1131 may be located near or adjacent the first end 1101. The airflow outlet 1610 may be located near or adjacent the second end 1102. The airflow channel 1110 may extend along the length of the assay 1300. Exhalation airflow 1112 may flow through the airflow inlet 1131, along the airflow channel 1110, and through the airflow outlet 1610. The housing 1100 may include a cavity 1640 adjacent the airflow inlet 1131. The cavity 1640 may be formed below the porous sample collection media 1120, as shown in FIG. 6D. The cavity 1640 may be in fluid communication with a channel 1630 extending along the length of the housing 1100. The channel 1630 may also house the assay 1300. The channel 1630 is further in fluid connection with the airflow outlet 1610.

The housing 1100 may be formed of two parts, a first part 1150 and a second part 1600. The first part 1150 may be a top part and the second part 1600 may be a bottom part. The airflow channel 1110 may be formed between the first and second parts 1150, 1600. The second part 1600 may include features that form the cavity 1640, the channel 1630, and the outlet 1610. For example, the second part 1600 may include a framework 1670 that facilitates such features. The second part 1600 may also include features, such as protrusions 1680, that facilitate coupling the second part 1600 with the first part 1150.

In the embodiment shown, the second part 1600 includes two holes for the airflow outlet 1610.

However, any number of holes could be used, such as 1, 2, 3, 4, 5, etc. The number and size of holes may be used to adjust resistance to flow through the airflow channel 1110.

The housing 1100 may also be constructed to facilitate housing liquid capsule 1210 including the metered dose of liquid 1201. When the housing 1100 is held horizontally, e.g., the length of the housing 1100 is held in a horizontal orientation with the airflow inlet 1131 facing upward, the opening 1220 of the liquid capsule 1210 may be positioned below the level of the porous sample collection media 1120. This may help control liquid flow from the liquid capsule 1210 to the porous sample collection media 1120 and to elute a captured sample from the porous sample collection media 1120 to the assay sample receiving area 1310. For example, positioning the liquid capsule 1210 below the level of the porous sample collection media 1120 may cause the liquid to wick through rather than to flood the porous sample collection media 1120.

The housing 1100 may include further liquid flow control features 1620, such as raised or indented features positioned below the liquid capsule 1210. In an exemplary embodiment, the features 1620 include a plurality of grooves 1621, shown in FIG. 7B. The grooves 1621 may be oriented in the desired direction of liquid flow-that is, toward the porous sample collection media 1120 and assay sample receiving area 1310. Other shapes are also possible, such as raised dimples, lines, or other shapes. The porous sample collection media 1120 may be disposed, in part, over the liquid flow control features 1620, and the liquid capsule 1210 may be disposed on top of the porous sample collection media 1120.

The liquid capsule 1210 may be separated from the porous sample collection media 1120 by the removable tab 1250. The removable tab 1250 may include a first portion 1251 and a second portion 1257. The first portion 1251 may be a sealing portion disposed against (e.g., sealed onto) the lip 1211 of the liquid capsule 1210. The first portion 1251 may be folded over itself and may include a fold 1252, as shown in FIG. 6D. The second portion 1257 may extend out from the housing 1100 and may form a pull tab.

The first and second parts 1150, 1600 may be connected by any suitable mechanism, such as a snap fit closure, friction fit, adhesive, heat seal, or the like. In some embodiments, the first and second parts 1150, 1600 may be connected by a tamper evident connection. Other possible tamper-indicating features include colored dye indicating user or tampering, or breakpoints (e.g., perforations, thin walls, torque breaking plastic portions, and/or spring like designs) that indicate to the user if the device has been used or tampered with.

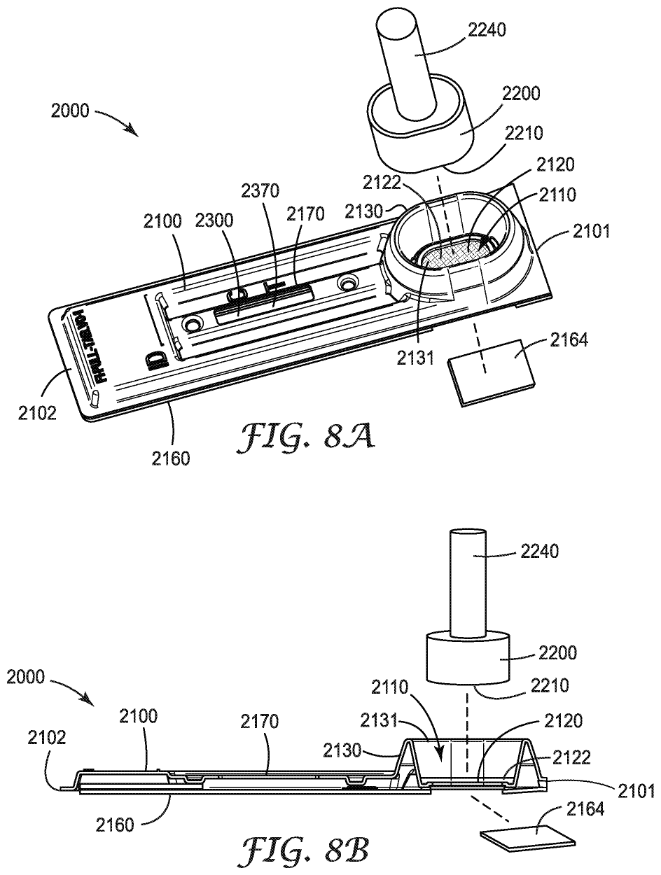

FIGS. 8A and 8B show an alternative embodiment of the sample collection and analysis system 2000, where the liquid reservoir 2200 is provided as a separate element to be received by the housing 2100. The system 2000 has a housing 2100 extending from a first end 2101 to a second end 2102. The housing 2100 has an air inlet 2131 constructed to receive an exhalation airflow. The air inlet 2131 may be provided as part of a mouthpiece 2130, as shown, or alternatively a nosepiece. A porous sample collection media 2120 is disposed within the housing. The housing 2100 defines an airflow path 2110 extending from the air inlet 2131 and through the porous sample collection media 2120. The porous sample collection media 2120 is constructed and arranged to capture viruses, other pathogens, or other analytes from exhalation airflow flowing through the airflow path 2110. The system 2000 further includes an assay 2300 disposed within the housing 2100 and constructed to receive an eluted sample from the porous sample collection media 2120. The assay 2300 may include a test result display 2370 to indicate the presence or absence of a virus or other pathogen or analyte. The housing 2100 may include a corresponding result viewing window 2170 through which the test result display 2370 may be viewed.

The liquid reservoir 2200 may include a capsule 2210 containing the metered dose of liquid. The liquid reservoir 2200 may further include a handle 2240 for convenient use and placement of the liquid reservoir 2200. The liquid reservoir 2200 may be received in the air inlet 2131 (e.g., mouthpiece 2130) of the housing 2100. The housing 2100 may include a grid or screen 2122 disposed within the air inlet 2131. The screen 2122 may be in the airflow path 2110 and cover the porous sample collection media 2120. The screen 2122 may act as a pre-filter as well as a surface that the liquid reservoir 2200 may be pressed against to rupture, puncture, peel, or otherwise break a seal on the capsule 2210 to release the metered dose of liquid. A user may hold the handle 2140 and place the capsule 2210 into the air inlet 2131. The user may then twist and/or press the capsule 2210 against the screen 2122 to release the metered dose of liquid onto the porous sample collection media.

The sample collection and analysis system 2000 may further include a removable and replaceable airflow outlet cover 2164. The airflow outlet cover 2164 may be removed from the bottom portion 2160 of the housing 2100 by a user to provide an airflow outlet in the bottom portion 2160. The user may further replace the airflow outlet cover 2164 to close the opening and to prevent liquid flow through the opening once the metered dose of liquid is applied onto the porous sample collection media 2120. Although the airflow outlet cover 2164 is shown as a separate piece, the airflow outlet cover 2164 may be connected to the bottom portion 2160. For example, the airflow outlet cover 2164 may be pivotably, slidably, hingedly, or peelably connected to the bottom portion 2160.

The screen 2122, the airflow outlet cover 2164, or both may optionally be incorporated into any of the embodiments discussed here, including those shown in FIGS. 1A, 4A, and 6A.

The sample collection and analysis systems discussed here may include an area for writing or otherwise indicating identifying information, such as a name, initials, account number, or the like.

The sample collection and analysis system may further include a machine-readable optical label. Such labels may include, for example, a bar code and a QR (quick response) code. The machine-readable optical label may be configured to display the result of the assay. The machine-readable optical label may be used to read and record the result. An electronic reader capable of reading machine-readable optical labels may be used to read and record the result. An electronic reader may be, for example, a smart phone, a tablet, a laptop, or bar code reader or QR code reader. The electronic reader may further be used to transmit the result, for example, to a healthcare provider or to a database.

A method of using the sample collection and analysis system may include exhaling into the air inlet (e.g., into the mouthpiece) to capture a sample in the porous sample collection media, forming a loaded porous sample collection media; at least partially removing (or moving) the removable tab to release a metered dose of liquid onto the porous sample collection media, thus eluting the sample from the loaded porous sample collection media, and allowing the eluted sample to flow onto the assay; and observing a test result in the result display of the assay. The method may further include reading the result display of the assay using an electronic reader.

The sample collection and analysis system may be provided as a kit. The kit may include the sample collection and analysis system as discussed above, and instructions for collecting a sample and testing the sample using the assay. The instructions may include instructions to: exhale along the airflow path to capture a sample in the porous sample collection media; move or remove the removable tab to release a metered dose of liquid onto the porous sample collection media; and observe a test result in a result display of the assay. The instructions may further include instructions to read a test result display of the assay using an electronic reader.

### Embodiments

The following is a list of exemplary embodiments according to the present disclosure.

Embodiment 1 is a sample collection system comprising: a housing comprising an air inlet constructed to receive an exhalation airflow; a porous sample collection media disposed within the housing; an airflow path extending from the air inlet through the porous sample collection media; a liquid reservoir in fluid communication with the porous sample collection media, constructed to direct liquid onto the porous sample collection media, the liquid reservoir comprising: a volume housing the liquid; an opening; and a removable tab sealing the opening; and an assay constructed to receive an eluted sample from the porous sample collection media.

Embodiment 2 is the sample collection system of embodiment 1, wherein the liquid reservoir is immediately adjacent to the air inlet.

Embodiment 3 is the sample collection system of embodiment 1 or 2, wherein the liquid reservoir is distanced from the air inlet.

Embodiment 4 is the sample collection system of embodiment 3, further comprising a liquid flow channel extending between the liquid reservoir and the porous sample collection media.

Embodiment 5 is the sample collection system of any preceding embodiment, wherein the removable tab is positioned between the volume and the porous sample collection media.

Embodiment 6 is the sample collection system of any preceding embodiment, wherein the removable tab is a pull tab.

Embodiment 7 is the sample collection system of any preceding embodiment, wherein the removable tab comprises a first portion disposed between the volume and the porous sample collection media and a second portion extending outside of the housing and forming a pull tab.

Embodiment 8 is the sample collection system of embodiment 7, wherein the first portion comprises a folded portion.

Embodiment 9 is the sample collection system of embodiment 7, wherein first portion is adhered to the liquid reservoir by a heat seal, an adhesive, a gasket, or a combination thereof.

Embodiment 10 is the sample collection system of any preceding embodiment, wherein the removable tab comprises perforations forming a break point.

Embodiment 11 is the sample collection system of any preceding embodiment, wherein the porous sample collection media defines a surface area and the volume of the liquid reservoir divided by the surface area is in a range from 10 μL/cm2 to 400 μL /m2, or from 10 μL/cm2 to 250 μL/cm2, or from 50 μL/cm2 to 150 μL/cm2.

Embodiment 12 is the sample collection system of any preceding embodiment, wherein the volume is in a range of 50 μL to 1000 μL.

Embodiment 13 is the sample collection system of any preceding embodiment, wherein the liquid reservoir houses from 50 μL to 1000 μL of the liquid. The metered dose of liquid may have a volume of 50 μL or greater, 100 μL or greater, 150 μL or greater, 200 μL or greater, or 250 μL or greater. The metered dose of liquid may have a volume of 1200 μL or less, 1000 μL or less, 750 μL or less, 500 μL or less, or 400 μL or less. The metered dose of liquid may have a volume of 50 μL to 1000 μL or 100 μL to 500 μL.

Embodiment 14 is the sample collection system of any preceding embodiment, wherein the liquid reservoir has a horizontal dimension parallel to the porous sample collection media and a vertical dimension orthogonal to the horizontal dimension, wherein the horizontal dimension is no more than 1 times the vertical dimension. The height (horizontal dimension) to width (vertical dimension) ratio may be 1 or less, 0.9 or less, 0.8 or less, or 0.75 or less. The height to width ratio may be 0.3 or greater, 0.4 or greater, or 0.5 or greater. The height to width ratio may be from 0.5 to 0.75.

Embodiment 15 is the sample collection system of any preceding embodiment, wherein the housing comprises a first end and an opposing second end, wherein the air inlet is disposed adjacent the first end, wherein an air outlet is disposed adjacent the second end, and wherein the airflow path extends from the air inlet to the air outlet.

Embodiment 16 is the sample collection system of embodiment 15, wherein the airflow path extends along a length of the assay.

Embodiment 17 is the sample collection system of any preceding embodiment, wherein the housing comprises a first part coupled with a second part, wherein the air inlet is formed in the first part.

Embodiment 18 is the sample collection system of embodiment 17, wherein the first part and second part are connected by a snap fit closure, and optionally wherein the snap fit closure is tamper evident.

Embodiment 19 is the sample collection system of any preceding embodiment, wherein the porous sample collection media comprises a nonwoven filtration layer having an electrostatic charge.

Embodiment 20 is the sample collection system of embodiment 19, wherein the nonwoven filtration layer is hydrophobic.

Embodiment 21 is the sample collection system of any preceding embodiment, wherein the liquid is an aqueous solution comprising a surfactant.

Embodiment 22 is the sample collection system of any preceding embodiment comprising two or more liquid reservoirs, each liquid reservoir constructed to deliver liquid onto the porous sample collection media, optionally wherein at least one of the two or more liquid reservoirs contains a liquid being different than a liquid contained in another of the two or more liquid reservoirs.

Embodiment 23 is the sample collection system of any preceding embodiment, further comprising a pre-filter fixed within the housing and along the airflow path and between the air inlet and the porous sample collection media.

Embodiment 24 is the sample collection system of any preceding embodiment, wherein the assay is constructed to detect presence of a virus or other pathogen in a collected sample.

Embodiment 25 is the sample collection system of any preceding embodiment, wherein the assay is a lateral flow assay, a vertical flow assay, or a colorimetric indicator.

Embodiment 26 is the sample collection system of any preceding embodiment, wherein the housing comprises a test result display window.

Embodiment 27 is a method of collecting and testing a sample, the method comprising: flowing exhalation air through a porous sample collection media to form a captured sample, the porous sample collection media being disposed within a sample collection device housing; releasing a metered dose of liquid onto the porous sample collection media from a liquid reservoir disposed within the sample collection device housing by pulling on a pull tab, the metered dose of liquid eluting the captured sample onto an assay disposed within the sample collection device housing; and observing a test result on the assay.

Embodiment 28 is the method of embodiment 27, wherein the pulling on the pull tab unseals an opening on the liquid reservoir.

Embodiment 29 is the method of embodiment 27 or 28, wherein the metered dose comprises from 50μL to 1000 μL of buffer. The metered dose of liquid may have a volume of 50 μL or greater, 100 μL or greater, 150 μL or greater, 200 μL or greater, or 250 μL or greater. The metered dose of liquid may have a volume of 1200μL or less, 1000 μL or less, 750 μL or less, 500 μL or less, or 400 μL or less. The metered dose of liquid may have a volume of 50 μL to 1000 μL or 100 μL to 500 μL.

Embodiment 30 is the method of any one of embodiments 27 to 29, wherein flowing exhalation air comprises blowing into a mouthpiece on the sample collection device.

Embodiment 31 is the method of any one of embodiments 27 to 29, wherein flowing exhalation air comprises blowing into a nosepiece on the sample collection device.

Embodiment 32 is the method of any one of embodiments 27 to 31, wherein the observing of the test result comprises observing a positive result if a target virus or other target pathogen is present or negative result if a target virus or other target pathogen is absent.

Embodiment 33 is the method of any one of embodiments 27 to 32, further comprising reading a QR code on the sample collection device.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A sample collection system comprising:
a housing comprising an air inlet constructed to receive an exhalation airflow;
a porous sample collection media disposed within the housing;
an airflow path extending from the air inlet through the porous sample collection media;
a liquid reservoir in fluid communication with the porous sample collection media, constructed to direct liquid onto the porous sample collection media, the liquid reservoir comprising:
a volume housing the liquid;
an opening; and
a removable tab sealing the opening; and
an assay constructed to receive an eluted sample from the porous sample collection media.

2-4.

5. The sample collection system of claim 1, wherein the removable tab is positioned between the volume and the porous sample collection media.

6. The sample collection system of claim 1, wherein the removable tab is a pull tab.

7. The sample collection system of claim 1, wherein the removable tab comprises a first portion disposed between the volume and the porous sample collection media and a second portion extending outside of the housing and forming a pull tab.

8. The sample collection system of claim 7, wherein the first portion comprises a folded portion.

9. The sample collection system of claim 7, wherein first portion is adhered to the liquid reservoir by a heat seal, an adhesive, a gasket, or a combination thereof.

10. The sample collection system of claim 1, wherein the removable tab comprises perforations forming a break point.

11. The sample collection system of claim 1, wherein the porous sample collection media defines a surface area and the volume of the liquid reservoir divided by the surface area is in a range from 10 μL/cm² to 400 μL/cm².

12. The sample collection system of claim 1, wherein the volume is in a range of 50 μL to 1000 μL.

13. (canceled)

14. The sample collection system of claim 1, wherein the liquid reservoir has a horizontal dimension parallel to the porous sample collection media and a vertical dimension orthogonal to the horizontal dimension, wherein the horizontal dimension is no more than 1 times the vertical dimension.

15. (canceled)

16. (canceled)

17. The sample collection system of claim 1, wherein the housing comprises a first part coupled with a second part, wherein the air inlet is formed in the first part, wherein the first part and second part are connected by a snap fit closure, and optionally wherein the snap fit closure is tamper evident.

18. (canceled)

19. The sample collection system of claim 1, wherein the porous sample collection media comprises a nonwoven filtration layer having an electrostatic charge.

20. The sample collection system of claim 19, wherein the nonwoven filtration layer is hydrophobic.

21. The sample collection system of claim 1, wherein the liquid is an aqueous solution comprising a surfactant.

22. The sample collection system of claim 1 comprising two or more liquid reservoirs, each liquid reservoir constructed to deliver liquid onto the porous sample collection media, optionally wherein at least one of the two or more liquid reservoirs contains a liquid being different than a liquid contained in another of the two or more liquid reservoirs.

23. The sample collection system of claim 1, further comprising a pre-filter fixed within the housing and along the airflow path and between the air inlet and the porous sample collection media.

24. The sample collection system of claim 1, wherein the assay is constructed to detect presence of a virus or other pathogen in a collected sample.

25. The sample collection system of claim 1, wherein the assay is a lateral flow assay, a vertical flow assay, or a colorimetric indicator.

26. The sample collection system of claim 1, wherein the housing comprises a test result display window.

27. A method of collecting and testing a sample, the method comprising:
flowing exhalation air through a porous sample collection media to form a captured sample, the porous sample collection media being disposed within a sample collection device housing;
releasing a metered dose of liquid onto the porous sample collection media from a liquid reservoir disposed within the sample collection device housing by pulling on a pull tab, the metered dose of liquid eluting the captured sample onto an assay disposed within the sample collection device housing; and
observing a test result on the assay.

28-31. (canceled)
